(19) United States
(12) Patent Application Publication
(10) Pub. No.: US 2022/0125651 A1
(43) Pub. Date: Apr. 28, 2022

(54) ANTISENSE OLIGOMERS FOR CONTROLLING CANDIDA ALBICANS INFECTIONS

(71) Applicant: UNIVERSIDADE DO MINHO, Braga (PT)

(72) Inventors: Sónia Carina MORAIS DA SILVA, Braga (PT); Daniela EIRA ARAÚJO, Barcelos (PT); Nuno Miguel MORAIS AZEVEDO, Lamego (PT); Mariana Contente RANGEL HENRIQUES, Braga (PT); Carina Manuela FERNANDES ALMEIDA, Braga (PT)

(21) Appl. No.: 17/430,137

(22) PCT Filed: Feb. 24, 2020
(86) PCT No.: PCT/IB2020/051552

§ 371 Date: Aug. 11, 2021

(30) Foreign Application Priority Data
Feb. 28, 2019 (PT) 115346
Mar. 1, 2019 (PT) 115349

(51) Int. Cl. A61F 13/84; C12N 15/113; C09D 5/14; C09D 199/00

(52) U.S. Cl. CPC A61F 13/8405; C12N 15/113; C09D 5/14; C09D 199/00; C12N 2320/31; C12N 2310/315; C12N 2310/341; C12N 2310/346; C12N 2310/11

(57) ABSTRACT

The present disclosure relates to the use of antisense oligomers in the treatment or therapy of Candida albicans infections. The present disclosure further describes the use of antisense oligomers in antisense therapy to inhibit the morphological transition of Candida albicans from yeast to filamentous form.

### TECHNICAL FIELD

The present disclosure relates to the use of antisense oligomers in the treatment or therapy of Candida albicans infections. In particular, the use of antisense oligomers to target specific genes involved in the morphological transition of Candida albicans from yeast to filamentous form.

The present disclosure further describes the use of antisense oligomers in antisense therapy to inhibit the morphological transition of Candida albicans from yeast to filamentous form.

### Sequence Listing

The application contains a Sequence Listing which has been submitted electronically in ASCII format and is hereby incorporated by reference in its entirety. Said ASCII copy, created on Jan. 5, 2022, is named 10224-010170-USO-v2_ST25.txt and is 3 KB in size.

### BACKGROUND

Candida species normally exist as commensal microorganisms but they can also act as opportunistic pathogens with the ability to cause superficial and systemic infections. The incidence of Candida infections has increased remarkably in the last few years due to the rise of the elderly population and the number of immunocompromised patients, as well as the widespread use of indwelling medical devices. Candida albicans infections remain as the most prevalent of all Candida species infections; approximately 47% in all Candida infections are caused by C. albicans.

As a dimorphic fungus, the ability of C. albicans to change from commensal to pathogenic is primarily due to its ability to morphologically switch between yeast and hyphal forms, a property that is central to its ability to penetrate human body tissues and escape the host's immune system. This ability contributes strongly to its pathogenicity. Candida albicans is responsible for causing about 400,000 deaths each year and one of its most problematic virulent factor is its ability to develop filaments.

In the last few years, important technical advances have facilitated the investigation of the molecular biology of C. albicans' morphological transition from yeast to filamentous form. These advances include the availability of genomic and transcriptomic sequence data essential for identifying and characterizing the genes involved in C. albicans' dimorphism. Several works have demonstrated the importance of EFG1, HWP1 and HYR1 genes as inducers of C. albicans filamentation [1].

Despite extensive research dedicated to the development of new therapeutic strategies, there are only a limited number of drugs available to fight superficial and invasive Candida infections. Indeed, only four molecular classes that target three distinct fungal metabolic pathways are currently used in clinics to treat Candida related systemic infections: polyenes, azoles, and echinocandins. Several other classes such as morpholines and allylamines are only used as topical agents due to either poor efficacy or severe adverse effects when administered systemically [2]. The increase in Candida multi-drug resistance, the scarcity of new drugs and the high plasticity of Candida species' transition switch from commensal to pathogenic fungi had led to the increase in the number of cases of candidiasis. In spite of the advances in therapies over the last few years, the rate of development of antifungal drugs continue to lag behind rate of fungal infections. Furthermore, most of these compounds have limited potential as systemic agents due to issues of toxicity.

These factors constitute a clinical problem, resulting in high morbidity and mortality (30-37%) as well as higher economic costs associated to prolonged hospital stays of patients. Average costs associated with candidemia among hospitalized patients range from €5700-€85000 per episode. These evidence highlight the need for new strategies to manage C. albicans infections.

Antisense therapy (AST) holds great promise for the treatment of many human diseases. The concept underlying AST is relatively straightforward: the use of a complementary sequence to a specific mRNA that can inhibit expression of the latter and induce a ‘blockage’ in translation of the DNA to protein. Antisense oligomers (ASO) are short strands of nucleic acids that are complementary to the target mRNA [16]. ASO generally compose of short sequences with 13-25 nucleotides of unmodified or chemically modified molecules that are gene specific. Moreover, in recent years, ASOs chemical modifications have been developed to enhance nuclease resistance, to prolong tissue half-live, affinity and potency, and to reduce specific toxicity. Currently there are three generations of ASOs. The first-generation contains phosphorothioate backbone modification and is characterized by the substitution of non-bridging phosphate oxygen with sulphur atoms. The second-generation was developed to enhance nuclease resistance and increase-binding affinity for target mRNA. For this 2′-O-methyl and 2′-O-methoexyethyl sugar modifications are added to the OH group in the 2′ position of the nucleotide. Numerous studies have documented the use of AST as biochemical tools for studying target human proteins [3]. This methodology has been proposed as an alternative to antibiotic treatments of bacteria infections. Fomivirsen (brand name Vitarvene), an S-oligo, is the only FDA-approved antisense therapeutic that targets a microorganism. Fomivirsen was approved in 1998 for treatment of cytomegalovirus-induced retinitis. Earlier work targeting bacteria found that modified ASOs inhibits growth of Salmonella, Listeria, Brucella, Pseudomonas, Staphylococcus, Streptococcus and Escherichia species [4]. Despite the fact that the development in ASO for use in controlling bacteria growth has been ongoing for the last decade, antisense based applications for use in controlling C. albicans growth is scarce and poorly exploited [5].

Document WO2014144024 (A1) discloses the method of producing fragments of the Candida cell Hyhr1 surface protein. The document further discloses the use of the protein for therapy and immunization against fungal infections.

Document AU2016244238 (A1) discloses the use of Hyhr1 surface protein as a target for immunization (active and passive) and as a prophylactic therapy for disseminate candidiasis.

Document US2019030141 (A1) discloses fragments of the Candida cell surface proteins Als3 and Hyr1 and combinations thereof for use in in immunization.

Document CN107304429 (A) discloses the use of inhibitors such as small molecule compounds to down-regulate Nuo2 protein or genes that inhibit virulence factor-related genes (ALS3, HWP1 and/or ECE1 gene expression).

Document US2017298349 (A1) discloses intergenic non-coding RNA molecules that regulate the expression of HWP1 and ALS3 of Candida. The document further discloses use of the non-coding RNA molecules and complementary molecules thereof in modulating HWP1 or ALS3 expression, as well as modulate the adherence, yeast-to-hyphal transition, or biofilm development of Candida. The aim of which is to prevent or treat candidiasis.

Document US2005244861 (A1) discloses nucleic acids required for the regulation of HWP1 expression in C. albicans and the use of these nucleic acids in identifying agents which inhibit the expression of HWP1.

Document CN1730654 (A) discloses the use of an ASO sequence for use against C. albicans infection. Specifically, the document discloses an ASO sequence against the core pseudoknot of C. albicans type I introne ribozyme thus strongly repressing the shearing reactivity of the C. albicans.

These facts are disclosed in order to illustrate the technical problem addressed by the present disclosure.

### General Description

This disclosure describes the use of ASOs to target specific genes involved in the morphological transition of C. albicans from yeast to filamentous form. The present disclosure further describes the use of ASOs in AST to inhibit the morphological transition of C. albicans from yeast to filamentous form.

ASOs are synthetic oligomers that silence expression of specific genes. This specificity confers an advantage over broad-spectrum antifungals by avoiding unintended effects on other commensal Candida species and, by minimizing the possibility of cross resistance. Furthermore, the sequence specificity and the short length of ASOs also pose little risk to human gene expression. Another important advantage of this approach is the ability to nearly eliminating or significantly reducing the time required for discovering new antifungals thus broadening the range of potentially available targets to any gene with a known base sequence in any yeast, for example C. albicans.

So far, there are no identified, designed and synthesized ASOs to target non-essential genes required for C. albicans virulence, namely genes involved in the morphological transition from yeast to filamentous form. The application of ASOs against a group of genes involved in one of most problematic virulence factor of C. albicans strains enable us to not only develop new nano-drugs specific for this problematic yeast but develop new strategies to control candidiasis. The present disclosure intends to identify short sequence ASOs that are able to specifically hybridize with the mRNA of three important regulator genes (EFG1, HWP1 and HYR1) of C. albicans filamentation and to block their molecular function. This allows for the development of nano-drugs to be used singly or in combination to control C. albicans' invasiveness and pathogenicity.

An aspect of the present disclosure relates to an isolated oligomer comprising: at least a sequence selected from a list consisting in the following sequences:
SEQ ID No. 7—for EFG1: 5′-mG mG mC mA TACCGTTA mU mU mG mU-3′;
SEQ ID No. 8—for HWP1: 5′ mUmGATAACATGTAATAAGmCmG 3′;
SEQ ID No. 9—for HYR1: 5′ mGmGmU TGA GAG TAmA mGmC 3′; or combinations thereof; or a variant of said sequence which is at least 95% identical to the selected sequence, based on the identity of all the nucleotides of said sequence, as a C. albicans filamentation blocker.

Another aspect of the present disclosure relates to an isolated oligomer comprising: at least a sequence selected from a list consisting in the following sequences: SEQ ID 7; SEQ ID 8; SEQ ID 9; or combinations thereof; or a variant of said sequence which is at least 95% identical to the selected sequence, based on the identity of all the nucleotides of said sequence, for use in medicine, preferably for use in the treatment or therapy of C. albicans related infections. In particular, for use in the treatment or therapy of vaginal infection and/or oral infections.

In an embodiment, the sequence is at least 96% identical to the selected sequence, based on the identity of all the nucleotides of said sequence; preferably 97%; 98%; 99% or identical.

Another aspect of the present disclosure relates to a composition comprising a combination of at least two isolated oligomers comprising: at least a sequence selected from a list consisting in the following sequences: SEQ ID 7; SEQ ID 8; SEQ ID 9; or combinations thereof; or a variant of said sequence which is at least 95% identical to the selected sequence, based on the identity of all the nucleotides of said sequence, as a controlling C. albicans filamentation blocker.

In an embodiment, the sequence is at least 96% identical to the selected sequence, based on the identity of all the nucleotides of said sequence; preferably 97%; 98%; 99% or identical.

In an embodiment, the combination of isolated oligomers is selected from the following combinations:
SEQ. ID. 7 and SEQ. ID. 8;
SEQ. ID. 7 and SEQ. ID. 9;
SEQ. ID. 8 and SEQ. ID. 9;
SEQ. ID. 7; SEQ. ID. 8 and SEQ. ID. 9.

In an embodiment, the composition may be a coating composition.

Another aspect of the present disclosure relates to an article comprising the isolated oligomer or the composition described in the present subject-matter, preferably a coating composition.

In an embodiment, the article may be a medical device, in particular a patch, a catheter, a stent, a contact lens or a pacemaker, among others.

In an embodiment, the article may be an intravaginal tampon, a sanitary napkin, or panty liners.

In one embodiment, ASOs targeting non-essential genes required for virulence, such as those that confer invasiveness and increases C. albicans pathogenicity were synthesized. It is considered that if in a pathogenic microorganism, the genetic sequence of a particular gene is known as a determinant agent of infection, synthesizing a strand of nucleic acid that will bind to the mRNA produced and inactivating it, in its translation into protein, it will be possible to control its virulence.

In another embodiment, cocktails of potential ASOs based on AST that are able to control C. albicans' morphological transition from yeast to filamentous form thus control the invasiveness of this microorganism were generated.

In one embodiment, the hybridization ability of ASOs with C. albicans cells were functionally analyzed. The ASOs' ability to inhibit the target genes' expression and their ability to reduce C. albicans' filamentation in different human body fluids were also functionally analyzed. These analyses were done using individual ASOs and combinations of ASOs.

### BRIEF DESCRIPTION OF THE DRAWINGS

The following figures provide preferred embodiments for illustrating the description and should not be seen as limiting the scope of invention.

FIG. 1 shows the antisense oligomers sequences according to the description.

FIG. 2 shows the anti-EFG1 and anti-HWP1 oligomers sensitivity and specificity against Candida species determined by fluorescence in situ hybridization assays.

FIG. 3 shows the cytotoxicity effect of 40 nM of anti-EFG1, anti-HWP1 and anti-HYR1 oligomers against 3T3 cell line (Fibroblast cells, Embryonic tissue, Mouse from CCL3, American Type Culture Collection).

FIG. 4 shows the effects of anti-EFG1, anti-HWP1 and anti-HYR1 oligomers on C. albicans filamentation.

FIG. 5 shows the effect of 40 nM of anti-EFG1 on C. albicans filamentation, on EFG1 gene expression and on efg1p translation at 24 h of incubation.

FIG. 6 shows the results related to different combinations with 40 nM of each oligomer (anti-EFG1, anti-HWP1 and anti-HYR1) on C. albicans filamentation during 24 h of incubation.

FIG. 7 shows the performance of anti-EFG1 oligomer on different human body fluids: artificial saliva (AS) and urine (AU) during 24 h and blood at 48 h of incubation.

### DETAILED DESCRIPTION

The present disclosure relates to the use of ASOs to target specific genes involved in the morphological transition of C. albicans from yeast to filamentous form.

The present disclosure further describes the use of ASO in AST to inhibit the morphological transition of C. albicans from yeast to filamentous form.

In one embodiment, ASOs targeting the three different genes were designed and synthetized to ensure the total blockade of C. albicans filamentation. The target regions were selected from each gene taking into account its high specificity against C. albicans genome and lower specificity against Homo sapiens genome. The regions selected were (5′-ACAATAACGGTATGCC-3′), (5′ CGCTTATTACATGTTATCA 3′) and (5′ GCTTACTCTCAACC 3′) for EFG1, HWP1 and HYR1, respectively.

In an embodiment, the target regions selected for methylation were:

SEQ ID No. 1-for EFG1:
5′-⁴⁷ACAATAACGGTATGCC⁶²-3′;
SEQ ID No. 2-for HWP1:
5′ ³³CGCTTATTACATGTTATCA⁵¹ 3′;
SEQ ID No. 3-for HYR1:
5′ ³⁶GCTTACTCTCAACC⁴⁹ 3′.

In one embodiment, for each sequence of the target regions selected the reverse complement was determined in order to design the respective ASOs. The sequences determined were (5′ GGCATACCGTTATTGT 3′), (5′TGATAACATGTAATAAGCG3′) and (5′GGTTGAGAGTAAGC 3′) for EFG1, HWP1 and HYR1, respectively.

The reverse complement sequences determined for methylation were:

SEQ ID No. 4-for EFG1:
5′ GGCATACCGTTATTGT 3′;
SEQ ID No. 5-for HWP1:
5′TGATAACATGTAATAAGCG3′;
SEQ ID No. 6-for HYR1:
5′GGTTGAGAGTAAGC 3′.

In one embodiment, in order to increase the ASOs hit-rate, part of the oligonucleotides belonging to each selected sequence were chemically modified based on second generation nucleic acid mimics design (2′-O-methyl).

In another embodiment, once it has been demonstrated that the inclusion of the two or more modifications in each end of the nucleic acid mimics increase its stability in human serum, antisense oligomers were designed and synthesized.

In one embodiment, anti-EFG1 oligomer was designed and synthetized with four 2′-O-methyl chemical modifications (5′-mG mG mC nnA TACCGTTA mU mU mG mU-3′). Anti-HWP1 oligomer was designed and synthetized with two chemical modifications (5′ mUmGATAACATGTAATAAGmCmG 3′). Anti-HYR1 oligomer was designed and synthetized with three chemical modifications (5′ mGmGmU TGA GAG TAmA mGmC 3′).

In an embodiment, the methylated sequences were:

SEQ ID No. 7-for EFG1:
5′-mG mG mC mA TACCGTTA mU mU mG mU-3′;
SEQ ID No. 8-for HWP1:
5′ mUmGATAACATGTAATAAGmCmG 3′;
SEQ ID No. 9-for HYR1:
5′ mGmGmU TGA GAG TAmA mGmC 3′.

Methods for the alignment of sequences for comparison are well known in the art, such methods include BLAST and FASTA. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). The sequence identity values, which are indicated in the present subject matter as a percentage were determined over the entire amino acid sequence, using BLAST with the default parameters.

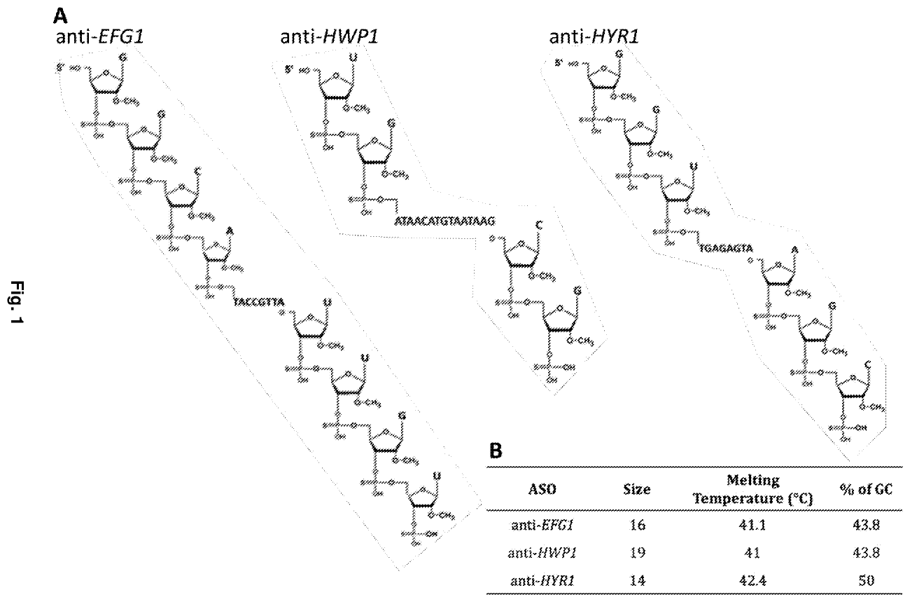

In an embodiment, FIG. 1 shows the ASOs sequences for EFG1 (anti-EFG1), HWP1 (anti-HWP1) and HYR1 (anti-HYR1) genes of C. albicans and the respective 2′-O-methyl chemical modifications insertions. FIG. 1B summarizes the characteristics of the three ASOs sequences in terms of its size, melting temperature and percentage of guanine and cytosine (GC).

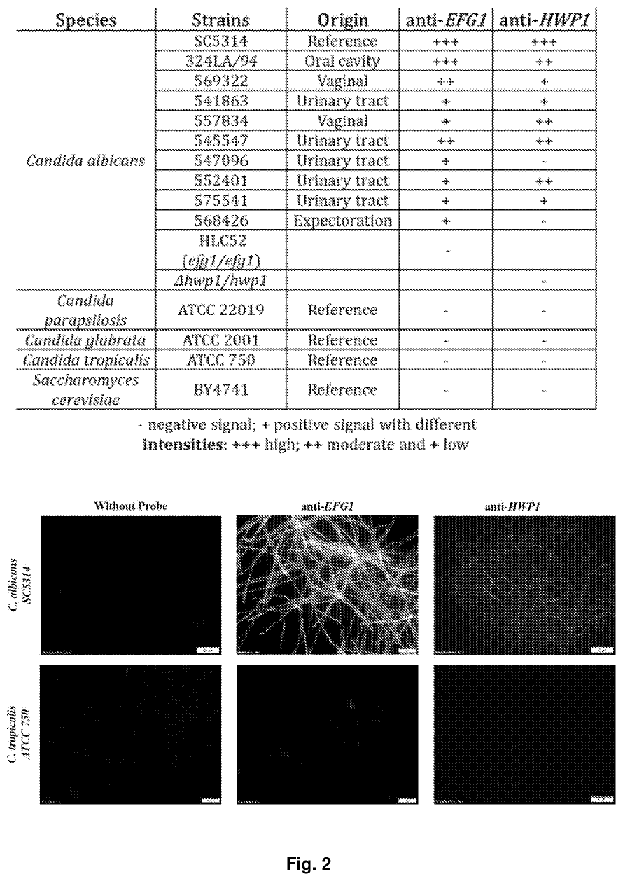

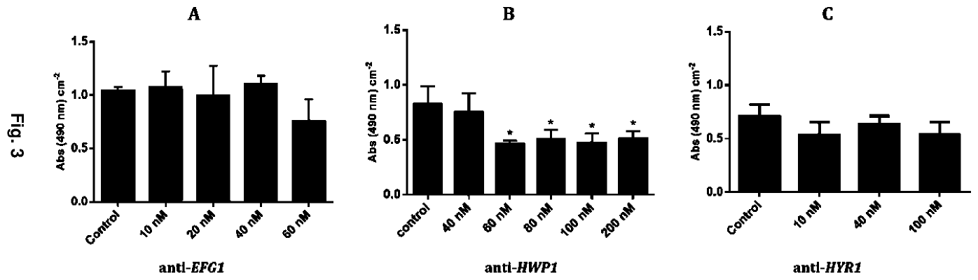

In an embodiment, FIG. 2 shows Anti-EFG1 and anti-HWP1 oligomers sensitivity and specificity against Candida species determined by fluorescence in situ hybridization assays. FIG. 2A summarizes the intensity of fluorescence obtained after 2 h of hybridization at 37° C. for different Candida species tested. FIG. 2B shows the fluorescence images of Candida species hybridization with anti-EFG1 and anti-HWP1 labelled with red fluorescein (56-FAM) and green fluorescein (TYE 563).

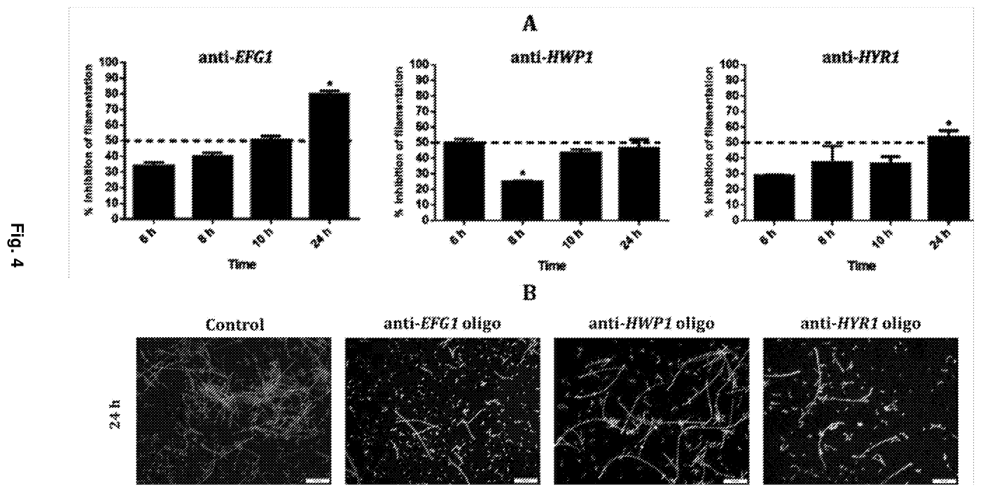

In an embodiment, FIG. 3 shows the cytotoxicity effects of 40 nM of Anti-EFG1, anti-HWP1 and anti-HYR1 oligomers against 3T3 cell line (Fibroblast cells, Embryonic tissue, Mouse from CCL3, American Type Culture Collection). These were measured using MTS kit (CellTiter 96® Aqueous One Solution Cell Proliferation Assay, Promega). The error bars represent standard deviation.

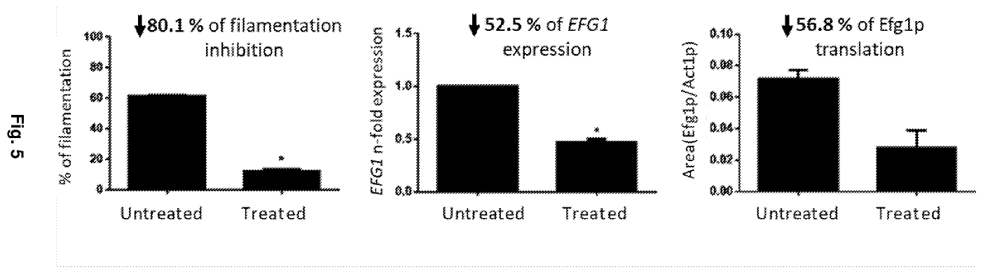

In an embodiment, FIG. 4 shows the effects of anti-EFG1, anti-HWP1 and anti-HYR1 oligomers on C. albicans filamentation. FIG. 4A shows the effect of 40 nM of each oligomer in terms of percentage of C. albicans filamentation inhibition. FIG. 4B shows the fluorescence images of C. albicans filamentation reduction when treated with the ASOs for 24 h. The control is related to C. albicans cultured in same conditions in absence of the oligomers. The error bars represent standard deviation. Statistical differences among the different time point tested (P<0.05) are marked with *.

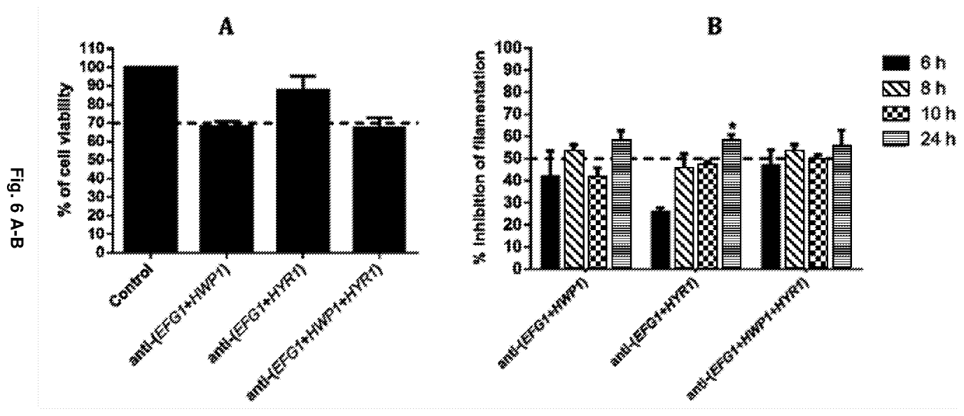

In an embodiment, FIG. 5A shows the effect of 40 nM of anti-EFG1 on C. albicans filamentation ability after 24 h of incubation. The effect on EFG1 gene expression is measured by qRT-PCR (as shown in FIG. 5B) and on efg1p translation obtained by nanoLC-MS/MS analysis (as shown in FIG. 5C). The error bars represent standard deviation. Statistical differences between C. albicans treated with anti-EFG1 oligomer and untreated (P<0.05) are marked with *.

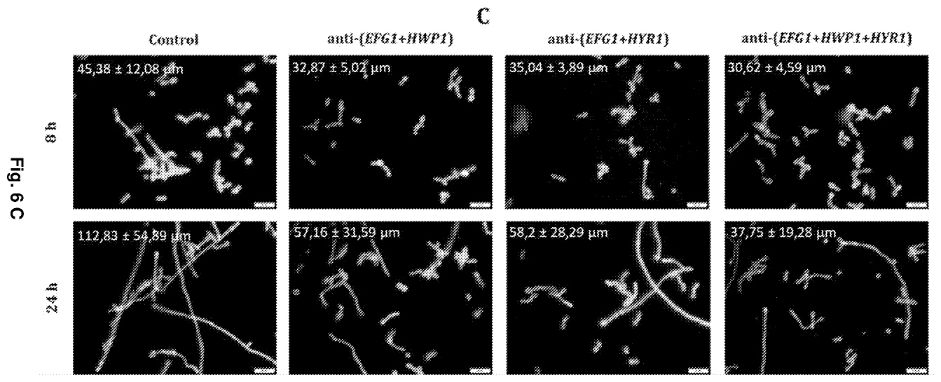

In an embodiment, FIG. 6 shows the results related with different combinations with 40 nM of each oligomer (anti-EFG1, anti-HWP1 and anti-HYR1). FIG. 6A shows the cytotoxicity effect against 3T3 cell line. This is measured using MTS kit. FIG. 6B shows the effect on C. albicans filamentation (% of inhibition). FIG. 6C shows the fluorescence images of C. albicans filamentation reduction at 8 h and 24 h of incubation. The error bars represent standard derivation. Statistical differences between C. albicans treated with mixed ASOs and untreated (P<0.05) are marked with *.

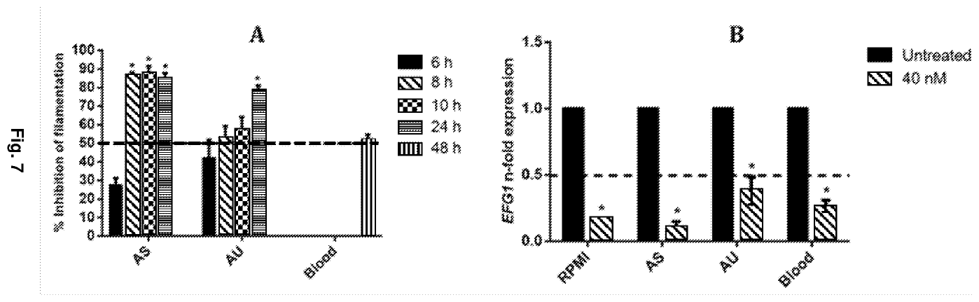

In an embodiment, FIG. 7 shows the performance of anti-EFG1 oligomer on human body fluids (AS-artificial saliva; AU-artificial urine and blood). FIG. 7A shows anti-EFG1 oligomer effect against C. albicans filamentation. FIG. 7B shows anti-EFG1 oligomer effect against EFG1 gene expression.

The above described embodiments are combinable.

The term “comprising” whenever used in this document is intended to indicate the presence of stated features, integers, steps, components, but not to preclude the presence or addition of one or more other features, integers, steps, components or groups thereof.

The following claims further set out particular embodiments of the disclosure.

### REFERENCES

Huang, G. (2012) Regulation of phenotypic transitions in the fungal pathogen Candida albicans. Virulence 3, 251-261
Silva, S. et al. (2017) Candida Species Biofilms' Antifungal Resistance. J. Fungi 3, 8 DeVos, S. L. and Miller, T. M. (2013) Antisense Oligonucleotides: Treating Neurodegeneration at the Level of RNA. Neurotherapeutics DOI: 10.1007/s13311-013-0194-5
Potaczek, D. P. et al. (2016) Antisense molecules: A new class of drugs. J. Allergy Clin. Immunol. 137, 1334-1346
Ecker et al., (1995). OLigonucleotides inhibiting Candida germ tube formation. U.S. Ser. No. 00/569,141A

1. An isolated oligomer comprising:
one or more sequences at least 95% identical to SEQ ID NO: 7; SEQ ID NO: 8; or SEQ ID NO: 9.

2. (canceled)

3. The isolated oligomer of claim 1, for use in the treatment or therapy of C. albicans related infections.

4. The isolated oligomer of claim 1, for use in the treatment or therapy of vaginal infection and/-or oral infections.

5. The isolated oligomer of claim 1, wherein the one or more sequences are at least 96% identical to SEQ ID NO: 7; SEQ ID NO: 8; or SEQ ID NO: 9.

6. A composition comprising at least two isolated oligomers, each isolated oligomer comprising:
a sequence at least 95% identical to SEQ ID NO: 7; SEQ ID NO: 8; or SEQ ID NO: 9.

7. The composition of claim 6, wherein the sequence is at least 96% identical to SEQ ID NO: 7; SEQ ID NO: 8; or SEQ ID NO: 9.

8. The composition according to of claim 6, comprising two or three isolated oligomers having the following sequences respectively:
SEQ ID NO: 7 and SEQ ID NO: 8;
SEQ ID NO: 7 and SEQ ID NO: 9;
SEQ ID NO: 8 and SEQ ID NO: 9; or
SEQ ID NO: 7; SEQ ID NO: 8 and SEQ ID NO: 9.

9. The composition according to of claim 6, wherein the composition is a coating composition.

10. An article comprising the composition of claim 6.

11. The article of claim 10, wherein the article is a medical device.

12. The article of claim 10, wherein the article is an intravaginal tampon, a sanitary napkin, or a panty liners.

13. The isolated oligomer of claim 1, comprising a sequence of SEQ ID NO: 7; SEQ ID NO: 8; or SEQ ID NO: 9.

14. The article of claim 10, wherein the article is a patch, a catheter, a stent, a contact lens or a pacemaker.

15. A method of inhibiting C. albicans filamentation, the method comprising contacting C. albicans with the isolated oligomer of claim 1.


SEQUENCE LISTING (9 sequences)

SEQ ID NO: 1 - DNA, 16 bp, Artificial Sequence
  EFG1
acaataacggtatgcc

SEQ ID NO: 2 - DNA, 19 bp, Artificial Sequence
  HWP1
cgcttattacatgttatca

SEQ ID NO: 3 - DNA, 14 bp, Artificial Sequence
  HYR1
gcttactctcaacc

SEQ ID NO: 4 - DNA, 16 bp, Artificial Sequence
  EFG1
ggcataccgttattgt

SEQ ID NO: 5 - DNA, 19 bp, Artificial Sequence
  HWP1
tgataacatgtaataagcg

SEQ ID NO: 6 - DNA, 14 bp, Artificial Sequence
  HYR1
ggttgagagtaagc

SEQ ID NO: 7 - DNA, 16 bp, Artificial Sequence
  Synthetic
  misc_feature: EFG1
  modified_base (1)..(4): 2′-O-Methyl modified nucleotide
  modified_base (13)..(16): 2′-O-Methyl modified nucleotide
ggcataccgttauugu

SEQ ID NO: 8 - DNA, 19 bp, Artificial Sequence
  Synthetic
  misc_feature: HWP1
  modified_base (1)..(2): 2′-O-Methyl modified nucleotide
  modified_base (18)..(19): 2′-O-Methyl modified nucleotide
ugataacatgtaataagcg

SEQ ID NO: 9 - DNA, 14 bp, Artificial Sequence
  Synthetic
  misc_feature: HYR1
  modified_base (1)..(3): 2′-O-Methyl modified nucleotide
  modified_base (12)..(14): 2′-O-Methyl modified nucleotide
ggutgagagtaagc